(19) United States
(12) Patent Application Publication
(10) Pub. No.: US 2011/0275602 A1
(43) Pub. Date: Nov. 10, 2011

(54) PHARMACEUTICAL ASSOCIATION, COMPOSITIONS FOR TOPICAL USE, FORMS OF DOSAGE AND METHOD OF TREATMENT OF ACUTE OR CHRONIC OTITIS IN PET ANIMALS

(76) Inventors: Jardel Massari, Ribeirao Preto (BR); Dolivar Coraucci Neto, Sertaozinho (BR)

(21) Appl. No.: 12/671,750

(22) PCT Filed: Aug. 18, 2008
(86) PCT No.: PCT/BR2008/000248

§ 371 Date: Jul. 15, 2011

(30) Foreign Application Priority Data
Aug. 22, 2007 (BR) PI 0703127-0

(51) Int. Cl. A61K 31/58; A61P 29/00; A61P 23/02; A61P 27/16; A61P 31/00

(52) U.S. Cl. 514/171

(57) ABSTRACT

The present invention refers to a new pharmaceutical association comprising antibiotic from the class of quinolones, azole antifungus, corticoid anti-inflammatory and local anesthesic from the class of amines. The pharmaceutical association of the present invention is particularly appropriate for the treatment of acute or chronic otitis in pet animals, caused by both bacteriae and fungi. The present invention also refers to compositions for topical use, as well as dosage forms comprising said pharmaceutical association, which are particularly appropriate for the treatment of acute or chronic otitis in cats and dogs.

### FIELD OF THE INVENTION

The present invention refers to a new pharmaceutical association comprising quinolinone antibiotic, azole antifungic, corticoid anti-inflammatory and local amine anesthesics compounds. The pharmaceutical association of the present invention is particularly appropriate to the treatment of acute or chronic otitis in pet animals, caused both by bacteriae such as Staphylococcus intermedius, Staphylococcus aureus, Staphylococcus spp, Streptococcus spp, Streptococcus pyogenes, Proteus mirabilis, Proteus vulgaris, Proteus spp, Pseudomonas aeruginosa, Escherichia coli, Corynebacterium spp, among others such as fungi, e.g. Mallassezia pachydermatis (Pityrosporum canis), Epidermophyton floccosum, Microsporum canis, Trichophyton rubrum, Trichophyton mentagrophytes, Candida spp and others.

The present invention also refers to composition for topical use, as well as dosage forms comprising said pharmaceutical association, which are particularly appropriate to the treatment of acute or chronic otitis in cats and dogs.

### BACKGROUND OF THE INVENTION

External otitis is an inflammation of soft tissue components in the external auditive passage. Said affection constitutes one of the most common and frustrating problems found in the medicine for small animals, since its treatment is difficult and has high recurrence probability. Otitis may be caused in a patient by various factors, which makes diagnostics and treatment become very difficult.

Predisposing factors facilitate inflammation by allowing a favorable environment for the survival of perpetuating factors. As examples of predisposing factors, it could be mentioned the conformation of the auditive passage, humidity in the passage, ear hairs, race predisposition, immunodeficiency syndromes, endocrine unbalances, iatrogenic auditive traumatism and obstructing diseases. Perpetuating factors sustain and worse inflammatory processes, which routes include channel occlusion, secretion of irritating factors, pH changes in the passage and formation of an infection focus. Examples include bacterial infections caused by Staphylococcus intermedius, Proteus mirabilis, Pseudomonas aeruginosa, Corynebacterium spp and Escherichia coli, and yeast infections caused by Malassezia pachydermatis.

Typical signs as presented by animals suffering said disease are e.g. head shaking, ear itching and scrubbing, pain around the ears or the head, bad odor, behavior changes, etc.

Initial otitis treatment, after an antibiogram is performed, includes the full cleaning of the external ear, control of the active inflammatory process and the use of commercial otological preparations, but said choice must be careful.

It is therefore clear that otitis in pet animals is a complex affection requiring some care in its treatment.

### Ciprofloxacin

It is known from the specialized literature that ciprofloxacin is a fluouroquinolone antibiotic of second generation, bactericide for inhibiting the replication and transcription of the bacterial DNA. The advantages of using fluoroquinolones in anti-inflammatory treatments are related to their quick bactericidal action against a wide verity of clinically important bacterial microorganisms. Fluoroquinolones are powerful, well tolerated by animals and have been given through a variety of routes. They are mainly active for otitis of animals caused by the bacteriae Staphylococcus sp, Streptococcus sp, Proteus sp, Pseudomonas aeruginosa, Escherichia coli, Corynebacterium spp and others.

Literature mentions that, in assays with humans using ciprofloxacin, an efficacy of 77% has been obtained for the treatment of acute external otitis. In another experiment, the efficacy of ciprofloxacin as linked to dexametasone was higher than the use of ofloxacin, about 90% against 78% for clinical cure, and about 92% against 81.8% for microbiological cure. A four-day period was also observed to end otorrhea with the use of ciprofloxacin as associated to dexametasone, against a six day period with the use of ofloxacin.

The joint administration of ciprofloxacin and fluocinolone improved results in cases of ostheitic and cholesteatomose forms, while chronical otitis requires the association of ciprofloxacin-based topical treatment with systemic antibiotic therapy.

As opposed to first generation quinolones, the clinical administration of fluoroquinolones produces resistant mutants in a still relatively low frequency. No bacterial resistance to quinolones has been so far verified through plasmids.

### Ketoconazole

Ketoconazole is the reference antifungus imidazole and, as such, one of the most widely used compounds of the family of azoles worldwide. It is indicated for administration by topic and oral routes, and may be widely distributed throughout the skin and subskin tissue, which makes it become efficient for the treatment of surface or systemic skin infections by fungi.

The main route for action of azole compounds is the inhibition of the enzyme lanostrel demethylase, which takes part in the biosynthesis of ergosterol, important for the integrity and maintainance of the function of fungus cell membrane. The alteration of fluidity and permeability of the cytoplasma membrane of the fungus reduces nutrient collection, causing the inhibition of fungus growth, originating morphological changes resulting in cell necrosis.

Among the etiologic agents responsible for otitis in animals, yeasts from the genus Malassezia have been known for more than a century. In veterinary medicine, the species Malassezia pachydermatis is usually pointed out as responsible for external otitis in carnivorous pet animals and, more recently, by various forms of dermatitis suffered by dogs with local or generalized skin diseases. There are also reports of isolation of other Malassezia species in the skin of cats.

Therapeutical options as traditionally used for the treatment of Malassezia affections include a few azole derivatives, notably ketoconazole, itraconazole and enylconazole, chlorhexidine or selenium sulphate. Various works have already been performed to detect in vitro susceptibility of Malassezia pachydermatis against a few antifungus agents.

Concerning local skin problems, topical treatment is usually sufficient, and the most widely used drugs in these cases are ketoconazole, chlorhexidine or selenium sulphate. In general cases, systemic treatment with ketoconazole or itraconazole is also required. Such treatments should last various weeks and may be linked to topical medication. The main advantage of systemic therapy is to eliminate yeasts as present in the skin and also in mucosae, considered by some authors as true reservoirs of said yeasts.

### Fluoroquinolone

Fluoroquinolone (acetonide) is a medicine included in the group of corticoids or corticosteroids, for administration to the skin or mucous membranes, with high anti-inflammatory power. Topical route is a common method of administration, since it reaches high drug concentrations in the site of action, while reducing the risks of systemic side effects.

Corticosteroids suffer just minimum absorption after being given on normal skin. There is a large regional anatomic variation in the penetration of corticosteroids and the solubility in the carrier represents important determination of the absorption of a topical steroid.

Glucocorticoids are able to block earlier manifestations of the inflammatory process, such as pain, heat and redness, up to later ones, such as tissue reparation and proliferation. Anti-inflammatory steroids affect all types of inflammatory responses, be them caused by invading pathogens, physical or chemical stimulation or inadequate immunological reaction. However, the exact nature of the action of glucocorticoids in pet animals has not been fully explained.

Effects of glucocorticoids involve interactions between steroids and intracell receivers, the latter ones found in nearly all tissues. After the interaction with the steroid, the receiver becomes “activated”, i.e. its conformation is changed, so to expose a DNA fixation dominiun. Said complex starts or avoids the transcription of some given genes.

Glucocorticoids inhibit many molecules linked to inflammation, such as cytokines, chemokines, metabolites of arachidonic acid and adherence molecules. They inhibit microvascular dilatation, oedema and permeability increase, hyperalgesia and fibrin deposition. They reduce the initial migration of polymorphonuclear leukocites and later leakage of monocytes, also reducing the phagocitary activity.

When topically applied, corticosteroids interact with receptor of dermal and intradermal cells. Said interaction induces peptides such as lipocurtains, which antagonize the action of A2 phospholipase, reducing the formation, release and action of endogenous chemical mediators of the inflammation. Such mediators include kinines, histamine, liposomal enzymes, prostaglandins and the complement system. Another effect contributing for the anti-inflammatory activity includes the stabilization of lisossomal and cell membranes, reduction of the number of Langerhans cells, inhibition of the movement of inflammatory cells, vessel constriction and antimitotic effect in various types of cells, including epidermal cells.

The vast majority of anti-inflamatories so far available for veterinary medicine is composed by active principles not presenting selective activity in inflammatory processes of the auditive system.

### Lidocaine

Lidocaine is a local anesthesic from the class of amides. It works at the central nervous system, cardiovascular system, respiratory tract, digestive tract, striated muscle fibers, urinary tract and reproductive tract. Lidocaine is a pharmaceutical with high affinity for body fats and adipose tissues.

Lidocaine is linked to plasma proteins, mainly alpha 1-acid glycoprotein. Said link has been reported to be highly variable and the concentrations depend and may be higher in dogs with an inflammatory disease.

Local anesthesics inhibit the generation and propagation of nervous impulses by blocking sodium channels, voltage-dependent in the nervous membrane. The most viable hypothesis is that the anesthesic enters the lipoprotein membrane and links itself to the receiving site at the sodium channel to avoid or forbid the ion movement. Currents as generated by sodium are reduced since the drug inhibits changes in channel conformation and therefore channels as linked to the drug no longer open. This delays the depolarization speed of the membrane, avoiding to reach the upper potential of the membrane. Blocking of sodium channels by most local anesthesics depend both on the voltage and frequency. Local anesthesics are able to block all nerves, thus their action is not limited to the loss of sensation, which is usually most desired, since motor loss also occurs. The disappearance of the nervous feature in response to local anesthesic blocking occurs in decreasing order, going through pain, heat, tact, deep pressure and finally motor function.

Surface or topical anesthesia results when the local anesthesic is applied to the skin or mucosae to cause loss of sensitivity by paralysis of sensorial nervous terminations. Local anesthesics reduce the frequency and rate of depolarization by increasing the upper limit value and reducing the permeability of the membrane to sodium. The exact site of action of anesthesics is the external surface of the sodium channel, the internal surface (axoplasmatic side) of the sodium channel inside the nervous cell membrane where the anesthesic produces side pressure, and the constriction of the sodium channel and the axoplasmatic face of the sodium channel, and inside the cell membrane.

### BRIEF DESCRIPTION OF THE INVENTION

Therefore, considering the complexity of otitis in pet animals and the background as reported above, the object of the present invention is to provide a new pharmaceutical association comprising one or more antibiotics from the class of quinolones, azole antifungus, corticoid anti-inflammatory and local anesthesic from the class of amines compounds, so to effectively cure said disease.

Another object of the present invention is to provide new compositions for topical administration, intended to the treatment of acute or chronic otitis in pet animals, particularly cats and dogs, mainly caused by the bacteriae Staphylococcus intermedius, Staphylococcus aureus, Staphylococcus spp, Streptococcus spp, Streptococcus pyogenes, Proteus mirabilis, Proteus vulgaris, Proteus spp, Pseudomonas aeruginosa, Escherichia coli, Corynebacterium spp, among others, and by fungi such as Mallassezia pachydermatis (Pityrosporum canis), Epidermophyton floccosum, Microsporum canis, Trichophyton rubrum, Trichophyton mentagrophytes, Candida spp and others.

Another particular object of the present invention is to provide new dosage forms for the topical administration of said compositions, as well as a method of treatment of acute or chronic otitis in pet animals, particularly cats and dogs.

### DETAILED DESCRIPTION OF THE INVENTION

These and other objects are reached by a new pharmaceutical association comprising one or more antibiotic compounds from the class of quinolones, azole antifungus, corticoid anti-inflammatory and local anesthesic from the class of amines. Said association is particularly intended for the topical treatment of acute or chronic otitis in pet animals, notably cats and dogs, caused by bacteriae such as Staphylococcus intermedius, Staphylococcus aureus, Staphylococcus spp, Streptococcus spp, Streptococcus pyogenes, Proteus mirabilis, Proteus vulgaris, Proteus spp, Pseudomonas aeruginosa, Escherichia coli, Corynebacterium spp and others, as well as fungi, such as Mallassezia pachydermatis (Pityrosporum canis), Epidermophyton floccosum, Microsporum canis, Trichophyton rubrum, Trichophyton mentagrophytes, Candida spp and others.

Antibiotics making part of the pharmaceutical association of the present invention are one or more from those pertaining to the group comprising ciprofloxacin, nalidixic acid, pyromidic acid, cynoxacin, rosoxacin, pipemidic acid, pefloxacin, ofloxacin, fleroxacin, enoxacin, difloxacin, amifloxacin, irloxacin, rufloxacin, lomefloxacin, norfloxacin, levofloxacin, trovafloxacin, temafloxacin, tosufloxacin, sparfloxacin, clinafloxacin and their respective pharmaceutically acceptable salts.

Preferably, the antibiotic is chosen from the group comprising ciprofloxacin, norfloxacin, levofloxacin, trovafloxacin and their pharmaceutically acceptable salts. Still more preferably, the antibiotic is ciprofloxacin and its pharmaceutically acceptable salts.

Salts from the pharmaceutically acceptable antibiotic compounds of the present invention are preferably hydrochlorides and lactates.

As azole antifungus agent of the association of the present invention, it may be selected one or more among compounds of the group ketoconazole, imidazole, chlotrimazole, econazole, myconazole, enylconazole, itraconazole, fluconazole, voriconazole, posaconazole, tioconazole, oxyconazole, isoconazole, bifonazole, sulconazole and ravuconazole.

Preferably, the azole antifungus agent is selected from the group comprising ketoconazole, imidazole, chlotrimazole, econazole, myconazole, enylconazole, itraconazole and fluconazole. Even more preferably, the azole antifungus compound is ketoconazole.

The corticoid anti-inflammatory of the pharmaceutical association of the present invention is chosen from the group comprising one or more from fluocinolone acetonide, chlobetasol, diflucortolone, halcinonide, halobetasole, alclometasone, amcinonide, beclometasone, betametasone, budesonide, clobetasone, desonide, deoxymetasone, diflorasone, diflucortolone, fluandrenolone, fluchlorolone, flumetasone, flucinolone, fluocinonide, fluocortin butyl, fluocortolone, fluprepnidene, halcinonide, halobetasol, hydrocortisone, methylprednisolone, mometasone, prednicarbate, tiamcinolone and their respective acceptable pharmaceutical salts.

Other corticosteroids which may integrate the pharmaceutical association of the present invention refer to the group comprising alclometasone dipropionate, amcinonide, beclometasone dipropionate, betametasone dipropionate, betametasone valerate, budesonide, chlobetasol propionate, clobetasone butyrate, desonide, deoxymetasone, difluorasone diacetate, diflucortolone valerate, fluandrenolone, fluchlorolone, flumetasone pivalate, fluocinolone valerate, fluocinonide, fluocortin butyl, fluocortolone hexanoate, fluocortolone pivalate, fluprednidene acetate, halcinonide, halobetasole propionate, hydrocortisone butyrate, hydrocortisone aceptonate, hydrocortisone acetate, methylprednisolone aceponate, mometasone furoate, prednicarbate, thiamcinolone acetonide.

Preferably, the corticoid anti-inflammatory of the present invention is selected from the group comprising fluocinolone, chlobetasol, diflucortolone, halcinonide, halobetasol, betametasone, budesonide, flumetasone, hydrocortisone, methylprednisolone and thiamcinolone. Even more preferably, the corticoid anti-inflammatory is fluocinolone.

Pharmaceutically acceptable salts from corticoid anti-inflammatory compounds are prefereably hydrochlorides.

As anesthesical compounds, the pharmaceutical association of the present invention comprises one or more as selected from the group comprising lidocaine, procaine, chlorprocaine, tetracaine, benzocaine, mepivacaine, prilocaine, ropivacaine, bupivacaine, etidocaine and their corresponding pharmaceutically acceptable salts. Lidocaine and its pharmaceutically acceptable salts, such as hydrochloride, sodium salt and carbonate, notably hydrochloride, are preferable as the anesthesic of the present invention.

Compositions for topical use object of the present invention typically comprise as their active ingredients, in percentage by weight, 0.03 to 3.0% of antibiotic from the class of quinolones, 0.1 to 10.0% of azole antifungus, 0.002 to 0.20% of corticoid anti-inflammatory and 0.2 to 20.0% of local anesthesic from the class of amines, and pharmaceutically acceptable excipients.

Particularly preferred are the compositions comprising the active ingredients, in percentuals by weight, 0.03 to 3.0% of ciprofloxacin, 0.1 to 10.0% of ketoconazol, 0.002 to 0.20% of fluocinolone and 0.2 to 20.0% lidocaine.

Even more particularly, compositions for topical use of the present invention comprise, in percentage by weight, 0.03 to 3.0% of ciprofloxacin hydrochloride, 0.1 to 10.0% of ketoconazole, 0.002 to 0.20% of fluocinolone hydrochloride and 0.2 to 20.0% of lidocaine hydrochloride.

Compositions for topical use of the present invention additionally comprise excipients in appropriate quantities to formulate the active ingredients. Said excipients should be generally pharmaceutically acceptable and include, among others, thickening agents, carries and coloring agents.

As examples of pharmaceutically acceptable thickening agents which may be used in the compositions of the present invention, we mention the group consisting of polyvinylpyrrolidone 90K, polyvinylpyrrolidone K-12, polyvinylpyrrolidone K-17, polyvinylpyrrolidone K-30, polyvinylpyrrolidone/vinyl acetate (6:4), hydroxyethylcellulose, methylcellulose, carboxymethylcellulose, hydroxypropylmethylcellulose, carboxyvinyl polymers or carbomers, polyacrylates, natural or synthetic gums, alginates, clays, polyoxyethylene glycols, gelatin, xanthan, pectins and pectates.

Examples of carriers which may be used are one or more compounds from the group consisting of glycerin, propylene glycol, mineral oil, water, isopropyl alcohol, ethyl alcohol, glyceroformol, polyethylene glycols of various molecular weights, hexylene glycol, sorbitol and vegetal oils of soy, canola, peanuts, corn, cotton or sesame.

Examples of coloring agents for use in the present invention may be selected from the group consisting of amaranthus red, sunset yellow, sunset yellow 6 (CI 15985), tartrazin yellow 5 (CI 19140), shining blue 1 (CI 42090), indigotin blue 2 (CI 70015), patent blue 5 (CI 42051), Bordeaux red 2 (CI 16185), erythrosin red 3 (CI 45430), azorubin red 5 (CI 14720), ponceaux red 6 4R (CI 16255), allura red 40 (CI 16035), pigments of iron oxide and titanium dioxide.

Furthermore, the invention refers to dosage forms for the pharmaceutical association and compositions of the present invention. Said dosage forms should guarantee the efficacy of the topical treatment of acute or chronic otitis in pet animals, particularly cats and dogs.

Thus, preferably, dosage forms of the present invention should comprise enough quantity of active ingredients to supply to the animal dosages of 0.001 to 10.0 mg/kg of body weight of antibiotic from the class of quinolones, 0.001 to 20.0 mg/kg of body weight of azole antifungus, 0.0001 to 10 mg/kg of body weight of corticoid anti-inflammatory and 0.001 to 20.0 mg/kg of body weight of local anesthesic from the class of amines. More preferably, dosage forms of the present invention comprise enough quantity of active ingredients to supply to the animal dosages of 0.001 to 10.0 mg/kg of body weight of ciprofloxacin, 0.001 to 20.0 mg/kg of body weight of ketoconazole, 0.0001 to 10 mg/kg of body weight of fluocinolone and 0.001 to 20.0 mg/kg of body weight of lidocaine.

Especially preferred are the dosage forms comprising sufficient quantity of active ingredients to supply the animal with dosages of 0.045 mg/kg of body weight of ciprofloxacin hydrochloride, 0.15 mg/kg of body weight of ketoconazole, 0.003 mg/kg of body weight of fluocinolone hydrochloride and 0.30 mg/kg of body weight of lidocaine hydrochloride.

### Examples

The present invention will be disclosed with reference to the examples that follow, which show various forms of embodiment of the invention, but not imposing any limit or restriction to the scope as defined by the set of claims.

Various products based on the association object of the present invention have been formulated, comprising active ingredients under the corresponding quantities as detailed below, based on 100 grams of product.

TABLE 1

| | Compound | | | |
|---|---|---|---|---|
| Formulation | A | Compound B | Compound C | Compound D |
| 1 | 0.3 g | 1.0 g | 0.02 g | 2.0 g |
| 2 | 0.15 g | 0.5 g | 0.01 g | 1.0 g |
| 3 | 0.6 g | 2.0 g | 0.04 g | 4.0 g |
| 4 | 0.9 g | 3.0 g | 0.06 g | 6.0 g |
| 5 | 1.2 g | 4.0 g | 0.08 g | 8.0 g |
| 6 | 0.03 g | 0.1 g | 0.002 g | 0.2 g |
| 7 | 3.0 g | 10.0 g | 0.2 g | 20.0 g |

Compound (A): ciprofloxacin (hydrochloride)
Compound (B): ketoconazol
Compound (C): fluocinolone hydrochloride
Compound (D): lidocaine hydrochloride

Formulations above was for topical administration on the external auditive passage, after its cleaning to remove all earwax excess and other dirts, under preferable dosages of four drops for dogs and three drops for cats, during seven to ten consecutive days. The treatment was not interrupted before 48 hours after symptoms disappear, which usually occurred after between ten and twenty days of treatment.

1. A pharmaceutical association for the treatment of acute or chronic otitis in pet animals, notably cats and dogs, comprising:
an antibiotic from the class of quinolones,
an azole antifungus,
a corticoid anti-inflammatory, and
a local anesthesic from the class of amines compounds.

2. The pharmaceutical association as claimed in claim 1, wherein the antibiotic is at least one antibiotic selected from the group consisting of ciprofloxacin, nalidixic acid, pyromidic acid, cinoxacin, rosoxacin, pipemidic acid, pefloxacin, ofloxacin, fleroxacin, enoxacin, difloxacin, amyfloxacin, irloxacin, rufloxacin, lomefloxacin, norfloxacin, levofloxacin, trovafloxacin, temafloxacin, tosufloxacin, sparfloxacin, clinafloxacin, and pharmaceutically acceptable salts thereof.

3. The pharmaceutical association as claimed in claim 2, wherein the antibiotic is ciprofloxacin or its pharmaceutically acceptable hydrochlorides or lactates.

4. The pharmaceutical association as claimed in claim 1, wherein the azole antifungus is at least one azole antifungus selected from the group consisting of ketoconazole, imidazole, clotrimazole, econazole, miconazole, enylconazole, itraconazole, fluconazole, voriconazole, posaconazole, tioconazole, oxyconazole, isoconazole, bifonazole, sulconazole, and ravuconazole.

5. The pharmaceutical association as claimed in claim 4, wherein the azole antifungus is ketoconazole.

6. The pharmaceutical association as claimed in claim 1, wherein the corticoid anti-inflammatory is at least one corticoid anti-inflammatory selected from the group consisting of fluocinolone acetonide, chlobetasol, diflucortolone, halcinonide, halobetasole, alclometasone, amcinonide, beclometasone, betametasone, budesonide, clobetasone, desonide, deoxymetasone, diflorasone, diflucortolone, fluandrenolone, fluchlorolone, flumetasone, flucinolone, fluocinonide, fluocortin butyl, fluocortolone, fluprepnidene, halcinonide, halobetasol, hydrocortisone, methylprednisolone, mometasone, prednicarbate, thiamcinolone, and their corresponding acceptable pharmaceutical salts.

7. The pharmaceutcal association as claimed in claim 1, wherein the corticoid anti-inflammatory is at least one corticoid anti-inflammatory selected from the group consisting of alclometasone dipropionate, amcinonide, beclometasone dipropionate, metametasone dipropionate, betametasone valerate, budesonide, chlobetasol propionate, clobetasone butyrate, desonide, deoxymetasone, difluorasone diacetate, diflucortolone valerate, fluandrenolone, fluchlorolone, flumetasone pivalate, fluocinolone valerate, fluocinonide, fluocortin butyl, fluocortolone hexanoate, fluocortolone pivalate, fluprednidene acetate, halcinonide, halobetasol propionate, hydrocortisone butyrate, hydrocortisone aceptonate, hydrocortisone acetate, methylprednisolone aceponate, mometasone furoate, prednicarbate, and thiamcinolone acetonide.

8. The pharmaceutical association as claimed in claim 6, wherein the corticoid anti-inflammatory is fluocinolone hydrochloride.

9. The pharmaceutical association as claimed in claim 1, wherein the anesthesic is at least one anesthesic selected from the group consisting of lidocaine, procaine, chloroprocaine, tetracaine, benzocaine, mepivacaine, prilocaine, ropivacaine, bupivacaine, etidocaine, and their corresponding pharmaceutical acceptable salts.

10. The pharmaceutical association as claimed in claim 9, wherein the anesthesic is lidocaine hydrochloride, sodium salt, or carbonate.

11. A composition for topical use for the treatment of acute or chronic otitis in pet animals, notably cats and dogs, comprising as active ingredients, in percentage by weight,
0.03 to 3.0% of an antibiotic from the class of quinolones,
0.1 to 10.0% of an azole antifungus,
0.002 to 0.20% of a corticoid anti-inflammatory, and
0.2 to 20.0% of a local anesthesic from the class of amines,
their pharmaceutically acceptable salts and pharmaceutically acceptable excipients.

12. The composition as claimed in claim 11, wherein the antibiotics from the class of quinolones, the azole antifungus, the corticoid anti-inflammatories, and the local anesthesics from the class of amines are those defined by any of claims 2 to 10.

13. The composition as claimed in claim 11, comprising as active ingredients, in percent by weight, 0.03 to 3.0% of ciprofloxacin, 0.1 to 10.0% of ketoconazol, 0.002 to 0.20% of fluocinolone, and 0.2 to 20.0% of lidocaine.

14. The composition as claimed in claim 11, comprising as active ingredients, in percent by weight, 0.03 to 3.0% of ciprofloxacin hydrochloride, 0.1 to 10.0% of ketoconazole, 0.002 to 0.20% of fluocinolone hydrochloride, and 0.2 to 20.0% of lidocaine hydrochloride.

15. A pharmaceutical association for the treatment of acute or chronic otitis in pet animals, notably cats and dogs, in a dosage form comprising sufficient quantity to supply the animal with 0.001 to 10.0 mg/kg of body weight of an antibiotic from the class of quinolones, 0.001 to 20.0 mg/kg of body weight of an azole antifungus, 0.0001 to 10 mg/kg of body weight of a corticoid anti-inflammatory, and 0.001 to 20.0 mg/kg of body weight of a local anesthesic from the class of amines.

16. The pharmaceutical association as claimed in claim 15, wherein the antibiotics from the class of quinolones, the azole antifungus, the corticoid anti-inflammatories, and the local anesthesics from the class of amines are those as defined by any of claims 2 to 10.

17. The pharmaceutical association as claimed in claim 15 in a dosage form comprising sufficient quantity of active ingredients to supply the animal with 0.001 to 10.0 mg/kg of body weight of ciprofloxacin as the antibiotic from the class of quinolones, 0.001 to 20.0 mg/kg of body weight of ketoconazol as the azole antifungus, 0.0001 to 10 mg/kg of body weight of fluocinolone as the corticoid antiinflammatory, and 0.001 to 20.0 mg/kg of body weight of lidocaine as the local anesthesic from the class of amines.

18. The pharmaceutical association as claimed in claim 15 in a dosage form comprising sufficient quantity of active ingredients to supply the animal with 0.045 mg/kg of body weight of ciprofloxacin hydrochloride as the antibiotic from the class of quinolones, 0.15 mg/kg of body weight of ketoconazole as the azole antifugus, 0.003 mg/kg of body weight of fluocinolone hydrochloride as the corticoid antiinflammatory, and 0.30 mg/kg of body weight of lidocaine hydrochloride as the local anesthesic from the class of amines.

19. A method for the treatment of acute or chronic otitis in pet animals, notably cats and dogs, comprising the application of a therapeutically effective quantity of a pharmaceutical association as defined by any of claims 1 to 10.
